(19) United States
(12) Patent Application Publication
(10) Pub. No.: US 2009/0118782 A1
(43) Pub. Date: May 7, 2009

(54) Complementary Configured Catheter Set for Intracardiac Recording and/or Pacing

(76) Inventor: Tristram D. Bahnson, Durham, NC (US)

Correspondence Address: MYERS BIGEL SIBLEY & SAJOVEC, PO BOX 37428, RALEIGH, NC, 27627, US

(21) Appl. No.: 11/997,962

(22) PCT Filed: Aug. 3, 2006
(86) PCT No.: PCT/US2006/030313

§ 371 Date: Nov. 25, 2008

Related U.S. Application Data
(60) Provisional application No. 60/705,867, filed on Aug. 5, 2005.

(51) Int. Cl. A61N 1/37

(52) U.S. Cl. 607/18; 607/25

(57) ABSTRACT

A catheter kit can include a pair of catheters, having different and complimentary electrode configurations each configured to provide access to different intraluminal electrophysiological monitoring and/or pacing sites during a procedure.

### RELATED APPLICATIONS

This application claims priority to U.S. Provisional Application 60/705,867, filed Aug. 5, 2005, the disclosure of which is incorporated by reference in its entirety.

### FIELD OF THE INVENTION

The invention relates to catheters for intracardiac electrocardiac recording and or pacing of a heart.

### BACKGROUND

Some diagnostic electrophysiological studies call for the simultaneous use of a number of multi electrode catheters to obtain intracardiac recordings, perform cardiac pacing, and guide therapy (e.g. catheter ablation). For example, catheter ablation for complex cardiac arrhythmias often requires placement of multiple intracardiac electrode catheters for recording intracardiac electrograms from various sites and/or pacing from any of multiple sites in each of the heart chambers. These catheters are sometimes referred to as diagnostic catheters. In some catheter ablation procedures, four different diagnostic catheters are used to record intracardiac electrograms from different locations, such as the right atrium, the right ventricle, left atrium, and inter-atrial septum/HIS bundle region. Additional diagnostic or non-diagnostic catheters may also be used, including an ablation catheter.

Accordingly, such procedures conducted using a relatively large number of catheters may be rather cumbersome. Furthermore, access to organs, such as the heart, may be limited by the number of blood vessels or pathways suitable for catheter insertion. The need to use multiple catheters for recording and pacing may limit availability of vascular access sites for other types of catheters such as intracardiac imaging catheters (e.g. intracardiac ultrasound), new mapping catheters (e.g, circular mapping catheters) or new therapeutic catheters (e.g. ablation catheters using cryotherapy or ablative ultrasound energy) that may be required during the same procedure.

### SUMMARY

According to some embodiments of the invention, a catheter kit includes a pair of catheters, having different and complimentary electrode configurations each configured to provide access to different intraluminal electrophysiological monitoring and/or pacing sites during a procedure.

According to some embodiments, a catheter kit includes a first catheter including a first electrode configuration to provide access to a plurality of first intraluminal electrophysiological monitoring and/or pacing sites via a first pathway during a procedure. A second catheter includes a second electrode configuration to provide access to a plurality of second intraluminal electrophysiological monitoring and/or pacing sites during the procedure.

According to some embodiments, a catheter kit includes a first catheter comprising a first plurality of electrodes on a distal portion of the first catheter having a spacing of 2-5-2 therebetween and comprising a second plurality of electrodes on a proximal portion of the first catheter having a spacing of about 8 m therebetween. The first and second pluralities of electrodes have a gap therebetween of about 33 mm. A second catheter includes a third plurality of electrodes on a distal portion of the second catheter having a spacing of 2-5-2 therebetween and a fourth plurality of electrodes on a proximal portion of the second catheter having a spacing of about 5 mm therebetween. The pluralities of first and second electrodes have a gap therebetween of about 25 mm.

In some embodiments, a catheter includes a catheter body configured for insertion into the heart during a procedure. The catheter body has a first plurality of electrodes at a proximal portion thereof and a second plurality of electrodes at a distal portion thereof. The first plurality of electrodes is configured to monitor electrophysiological signals and/or pace at an inter-atrial septum/HIS bundle region of a heart and the second plurality of electrodes is configured to monitor electrophysiological signals and/or pace at a right ventricle of the heart. In particular embodiments, at least one additional electrode is spaced apart from the first plurality of electrodes by a second gap opposite the second plurality of electrodes.

### BRIEF DESCRIPTION OF THE DRAWINGS

FIG. 1a is a schematic diagram illustrating a kit of two catheters inserted into the heart of a patient during a procedure according to some embodiments of the current invention;

FIGS. 1b-1c are fluoroscopic images of the kit of catheters of FIG. 1a shown in use; and

FIGS. 2-4 are schematic diagrams of catheters according to some embodiments of the current invention.

### DESCRIPTION OF EMBODIMENTS ACCORDING TO THE INVENTION

Embodiments of the present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

In some embodiments according the invention, catheters and/or a catheter kit can allow the acquisition of intracardiac electrode recordings and/or delivery of pacing stimuli which may otherwise require the use of additional catheters.

In particular embodiments, catheters in a two catheter kit can have a specific configuration of electrodes thereon. Moreover, the configuration of each of the catheters may be complementary to one another. Two “complementary” catheters may each provide a function that is different from the function(s) provided by the other complementary catheter. A “kit” can be sold together and/or may include instructions concerning how to use the catheters together, for example, as a complementary pair.

In certain embodiments, a catheter includes a catheter body configured for insertion into the heart during a procedure. The catheter body has a first plurality of electrodes at a proximal portion thereof and a second plurality of electrodes at a distal portion thereof. The first and second plurality of electrodes are configured to monitor electrophysiological signals and/or pace at specific cardiac regions.

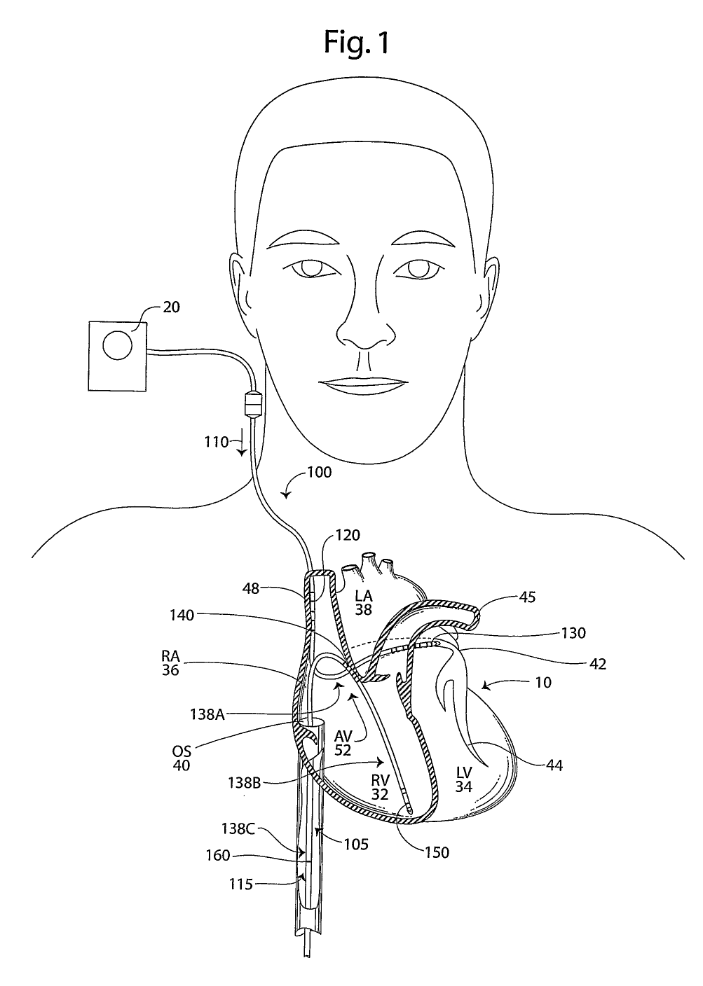

As illustrated in FIG. 1A, a heart 10 includes the right ventricle “RV” 32, the left ventricle “LV” 34, the right atrium “RA” 36, the left atrium “LA” 38, the superior vena cava 48, the atrioventricle junction “AV” or inter-atrial septum/HIS bundle region 52, the coronary sinus “CS” 42, the great cardiac vein 44, the left pulmonary artery 45, and the coronary sinus ostium or “os” 40. A first catheter 100 in the kit can be configured for use in a first part of the heart (accessed via a first pathway 110), whereas a second catheter 105 may be configured for use in a second part of the heart (accessed via a second pathway 115). The catheters 100, 105 can be controlled and/or electrophysiological information can be displayed on a controller/monitor (such as controller/monitor 20 connected to catheter 100). Accordingly, each of the catheters 100, 105 may be configured with a different number and/or placement of electrodes depending on which areas are to be treated and depending on how the catheter is to be inserted into the body. Furthermore, in some embodiments according to the invention, the spacing between the electrodes on the catheters may be selected with the specific use and/or pathway in mind so that the spacing allows access to the desired sites within the heart in a convenient way and to provide complementarity between the catheters in the kit.

Although the catheters 100, 105 are illustrated as being a two catheter kit, it should be understood that the catheters 100, 105 may also be employed separately. The catheters 100, 105 are configured for insertion into the body during a procedure, such as a cardiac ablation procedure for treatment of cardiac arrhythmias. After the procedure is completed, the catheters 100, 105 may be removed from the body. As shown in FIG. 1, the catheter 100 is inserted into the right internal jugular vein and the catheter 105 is inserted into the right femoral vein; however, it should be understood that other suitable access points may be used.

With continued reference to FIG. 1, the catheter 100 includes a group of electrodes 120 at a proximal portion of the catheter 100 and another group of electrodes 130 at a distal portion of the catheter 100. The two groups of electrodes 120, 130 are spaced apart from one another by a gap 138A. As illustrated, the catheter 100 is positioned in the coronary sinus 42 and the right atrium 36. In this configuration, the group of electrodes 120 can record electrical activity and/or pace within the coronary sinus 42 (which can include pacing and/or recording electrical activity for the left atrium 38 and/or left ventricle 34 of the heart 10) and the group of electrodes 130 can record electrical activity and/or pace the high lateral or lateral portion of the right atrium 36 of the heart 10.

The catheter 105 includes a group of electrodes 140 at a proximal portion of the catheter 105 and another group of electrodes 150 at a distal portion of the catheter 105. The two groups of electrodes 140, 150 are spaced apart from one another by a gap 138B. As shown, the catheter 105 is positioned in the right atrium 36 and is depicted over the catheter 100 and in the right ventricle 32. In this configuration, the group of electrodes 140 can record electrical activity and/or pace at the atrioventricle junction or inter-atrial septum/HIS bundle region 52 of the heart 10 and the group of electrodes 150 can record electrical activity and/or pace the right ventricle 32 of the heart 10. The catheter 105 can include at least one optional electrode 160, which may be used for complex ventricular ablation for unipolar pacing and/or recording electrical activity.

Although the catheters 100 and 105 are illustrated with respect to groups of electrodes 120, 130, 140 and 150 that are configured to record electrical activity and/or pace at the right atrium 36, left atrium 38, inter-atrial septum/HIS bundle region 52 and the right ventricle 32, respectively, it should be understood that other electrode configurations can be used to record electrical activity and/or pace in various other regions of the heart.

Images of the catheters 100 and 105 are shown in FIGS. 1B-1C.

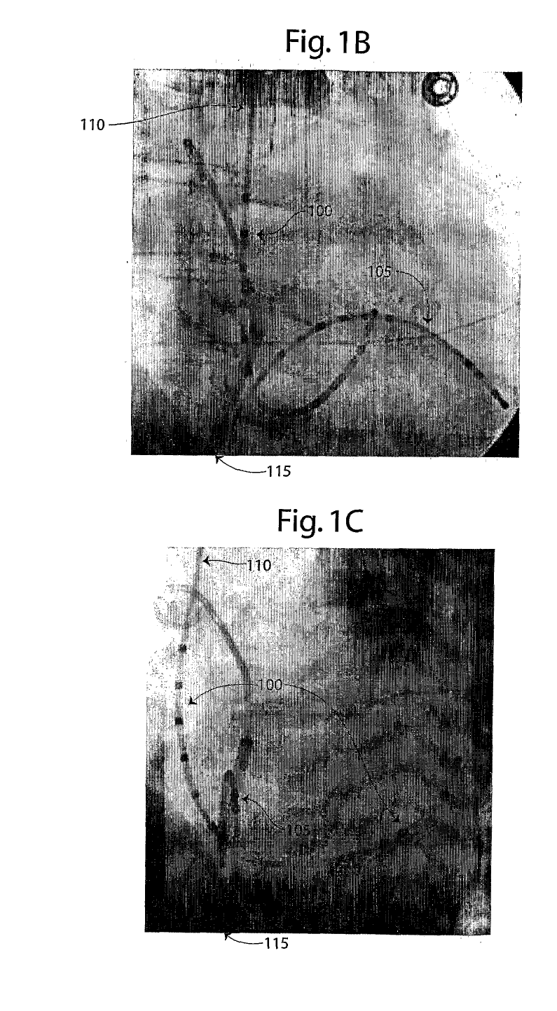

Embodiments according to the present invention are illustrated with respect to catheters 200, 305 and 405 shown in FIGS. 2, 3 and 4, respectively. It should be understood that the catheters 200, 305 and 405 can be used individually or in combination with one or more other catheters. In particular embodiments according to the invention, the catheters 200 and 305 can be used as a complementary pair such that the catheter 200 may be positioned to record electrical activity and/or pace at the right atrium 36 and left atrium 38, and the catheter 305 may be positioned to record electrical activity and/or pace in the inter-atrial septum/HIS bundle region 52 and the right ventricle 32. In certain embodiments according to the invention, the catheters 200 and 405 can be used as a complementary pair. That is, the catheter 200 may be positioned to record electrical activity and/or pace at the right atrium 36 and the left atrium 38, and the catheter 405 may be positioned to record electrical activity and/or pace in the inter-atrial septum/HIS bundle region 52 and the right ventricle 32. In addition to providing an additional electrode or pair of electrodes for unipolar pacing or recording.

As shown in FIG. 2, in some embodiments according to the invention, a catheter 200 can include a group of electrodes 220 at a proximal portion of the first catheter 200, and another group of electrodes 230 at a distal portion of the first catheter 200. The group of electrodes 220 can be spaced apart from the group of electrodes by a gap 238. The group of electrodes 220 can be spaced apart from one another by a first spacing 225 and the group of electrodes 230 can be spaced apart from one another by a second spacing A and/or B. For example, in some embodiments according to the invention, electrodes of one group of electrodes can be spaced apart to allow left atrial recordings accessed via the coronary sinus (i.e., a pathway), whereas the other group can be spaced apart to obtain recordings from the high lateral or lateral right atrium.

In some embodiments according to the invention, the gap 238 can be about 33 mm. It will be understood that the gap 238 may be free of other electrodes or alternatively, any electrodes located within the gap may be unused. In some embodiments according to the invention, the first group of electrodes 220 includes six electrodes arranged with between 5 and 10 mm spacing from one another, or in particular, an 8 mm spacing from one another. In other embodiments, the first group of electrodes 220 includes ten electrodes. In some embodiments according to the invention, the second group of electrodes 230 includes ten electrodes arranged in a 2-5-2 mm spacing. In particular, the electrodes with the second group 230 can be arranged in pairs having a spacing of about 2 mm between them (see B in FIG. 2). The pairs are spaced apart by about 5 mm (see A in FIG. 2). In some embodiments, the electrodes described herein are 2 mm wide metal electrodes formed from a combination of platinum and/or iridium.

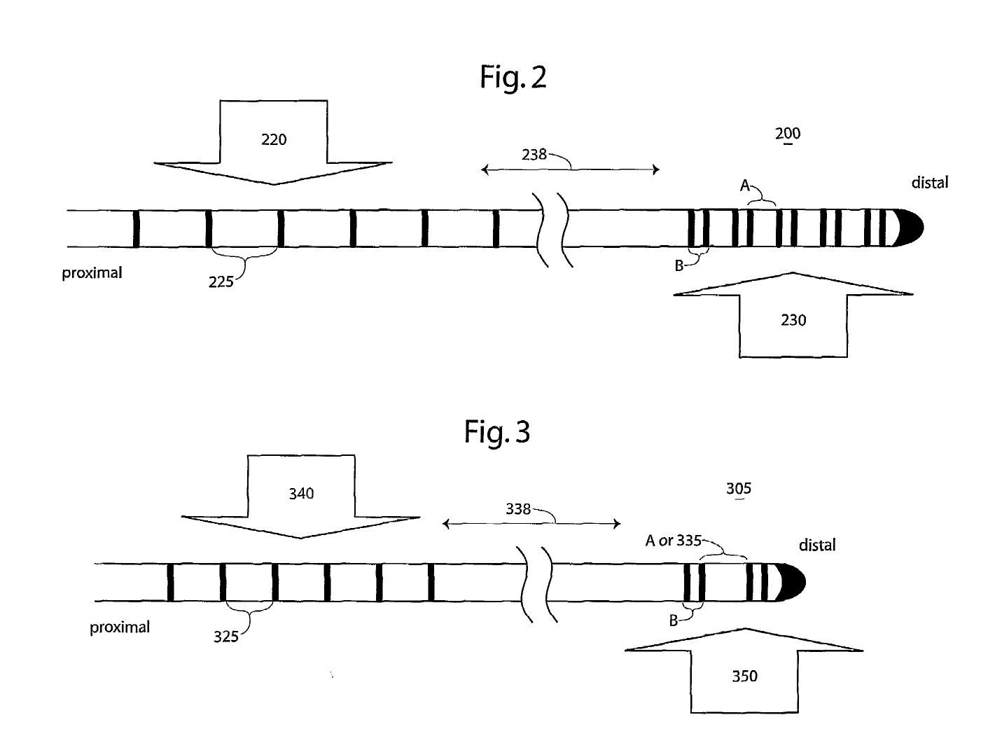

As shown in FIG. 3, in some embodiments according to the invention, a catheter 305 can include a group of electrodes 340 at a proximal portion of the catheter 305, and another group of electrodes 350 at a distal portion of the catheter 305. The group of electrodes 340 can be spaced apart from the group of electrodes 350 by a gap 338, which can be about 25 mm, or larger for patients with large hearts (e.g., 35 mm). The group of electrodes 340 can be spaced apart from one another by a spacing 325 and the group of electrodes 350 can be spaced apart from one another by a spacing 335 or alternate spacings A and B. It will be understood that the gap 338 may be free of other electrodes or alternatively, any electrodes located within the gap may be unused.

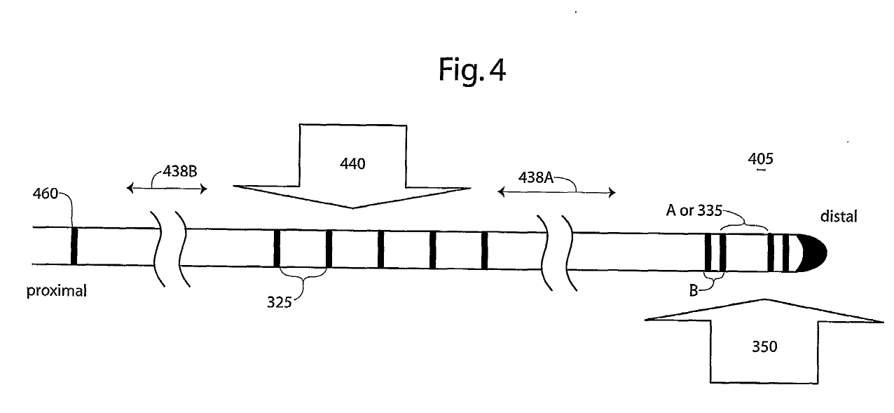

Referring to FIG. 4, in some embodiments according to the invention, a catheter 405 can include a group of electrodes 440 at a proximal portion of the catheter 405, and another group of electrodes 450 at a distal portion of the catheter 405. A single electrode 460 (or a pair of electrodes, for example, spaced apart from one another by 5 mm) can be positioned at a most proximal portion of the catheter 405. In some embodiments, two, three or more electrodes can be used for the electrode 460. The group of electrodes 440 can be spaced apart from the group of electrodes 450 by a gap 438A, and the group of electrodes 440 can be spaced apart from the electrode 460 by another gap 438A. The group of electrodes 440 can be spaced apart from one another by a spacing 425 and the group of electrodes 450 can be spaced apart from one another by a spacing 435 or alternate spacings A and B. It will be understood that the gaps 438A, 438B may be free of other electrodes or alternatively, any electrodes located within the gap may be unused.

It will be understood that the gap between the groups of electrodes can be selected based on a particular anatomical characteristic of a group of patients. For example, the gap 238 for a catheter used to treat patients with relatively large hearts may be more than 33 mm. In contrast, the gap for a catheter used to treat patients with relatively small hearts (such as children) may be less than 33 mm. In addition, the gaps 238 and 338 may be different from one another in order to obtain the desired placement of the groups of electrodes 220, 230, 340, and 350. For example, the gap 238 can be about 25-50 mm, or in particular, about 33 mm, and the gaps 338, 438A can be about 15-35 mm, or in particular, about 25 mm. The gap 438B can be about 10-40 cm, or in particular, about 25 cm or 30 cm such that the electrode 460 is positioned in the iliac vein or inferior vena cava vein, for example, for unipolar pacing and/or recording, such as is shown in FIG. 1A with respect to the electrode 160.

In some embodiments according to the invention shown in FIG. 3, the group of electrodes 340 includes 6 electrodes arranged with a 5 mm spacing from one another. In some embodiments according to the invention, the group of electrodes 350 includes 4 electrodes arranged with a 2-5-2 spacing from one another. In some embodiments according to the invention as shown in FIG. 4, the group of electrodes 440 includes 5 electrodes arranged with a 5 mm from one another, and the group of electrodes 450 includes 4 electrodes arranged with a 2-5-2 spacing from one another. In still further embodiments according to the invention, the electrodes may have different or irregular spacings than those described above in reference to FIGS. 2, 3 and 4. In some embodiments according to the invention, the electrode spacing may be selected based on a desired operation of the catheter.

As described above, the catheter 200 shown in FIG. 2 can be configured for use in a first part of the heart (accessed via, for example, the first pathway 110 of FIG. 1A), whereas the catheters 305, 405 shown in FIGS. 3 and 4 may be configured for use in a second part of the heart (accessed via, for example, the second pathway 115 of FIG. 1A). For example, the catheter 200 can be used to record and/or pace the right atrial/coronary sinus (RA/CS) and can be inserted via the right internal jugular vein. The catheters 305, 405 can be used to record and/or pace from a plurality of regions, for example, the right ventricle and inter-atrial septum/HIS bundle region via a catheter inserted into the heart via the right femoral vein. In still further embodiments according to the invention, the two catheters are configured to provide complimentary recordings, but access, for example, the heart via the same blood vessel (i.e., pathway).

Although embodiments according to the invention are illustrated with respect to two catheters, such as the catheter pair 100, 105, the catheter pair 200, 305, or the catheter pair 200, 405 it should be understood that a single catheter may be used or three or more complementary catheters may be used.

It will be understood that when an element is referred to as being “on”, “connected to” or “coupled to” another element, it can be directly on, connected or coupled to the other element or intervening elements may be present. In contrast, when an element is referred to as being “directly on,” “directly connected to” or “directly coupled to” another element, there are no intervening elements present. As used herein, the term “and/or” includes any and all combinations of one or more of the associated listed items. In the figures, the thickness and/or sizes of certain lines, layers, components, elements or features may be exaggerated for clarity and/or may not be drawn to scale.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention may be described herein with reference to illustrations that are schematic illustrations of idealized embodiments of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the drawings and specification, there have been disclosed typical embodiments of the invention and, although specific terms are employed, they are used in a generic and descriptive sense only and not for purposes of limitation, the scope of the invention being set forth in the following claims.

1. A catheter kit comprising:
a pair of catheters, having different and complimentary electrode configurations each configured to provide access to different intraluminal electrophysiological monitoring and/or pacing sites during a procedure.

2. The catheter kit according to claim 1, wherein a first catheter of the pair of catheters includes a first plurality of electrodes at a proximal portion thereof and a second plurality of electrodes at a distal portion thereof, wherein the first plurality of electrodes is configured to monitor electrophysiological signals and/or pace at a right atrium of a heart and the second plurality of electrodes is configured to monitor electrophysiological signals and/or pace within the coronary sinus.

3. The catheter kit according to claim 2, wherein the first and second plurality of electrodes are spaced apart by a first gap.

4. The catheter kit according to claim 3, wherein the first gap is between about 25 and 50 mm.

5. The catheter kit according to claim 2, wherein the first plurality of electrodes are spaced apart from one another by a first spacing pattern and the second plurality of electrodes are spaced apart from one another by a second spacing pattern, and the first spacing pattern is different from the second spacing pattern.

6. The catheter kit according to claim 5, wherein the first spacing pattern includes at least six electrodes arranged between about 5 and 10 mm from one another.

7. The catheter kit according to claim 5, wherein the second spacing pattern includes at least ten electrodes comprising five pairs of electrodes having a spacing of about 2 mm between each of the electrodes in a pair, and each pair of electrodes are spaced apart by about 5 mm from an adjacent pair.

8. The catheter kit according to claim 2, wherein the first catheter is configured for insertion via the right internal jugular vein.

9. The catheter kit according to claim 2, wherein a second catheter of the pair of catheters includes a third plurality of electrodes at a proximal portion thereof and a fourth plurality of electrodes at a distal portion thereof, wherein the third plurality of electrodes is configured to monitor electrophysiological signals and/or pace at an inter-atrial septum/HIS bundle region of a heart and the fourth plurality of electrodes is configured to monitor electrophysiological signals and/or pace at a right ventricle of the heart.

10. The catheter kit according to claim 9, wherein the third and fourth plurality of electrodes are spaced apart by a second gap.

11. The catheter kit according to claim 10, wherein the second gap is between about 15 and 35 mm.

12. The catheter kit according to claim 9, wherein the third plurality of electrodes are spaced apart from one another by a third spacing pattern and the fourth plurality of electrodes are spaced apart from one another by a fourth spacing pattern, and the third spacing pattern is different from the fourth spacing pattern.

13. The catheter kit according to claim 12, wherein the third spacing pattern includes at least six electrodes arranged about 5 mm from one another.

14. The catheter kit according to claim 12, wherein the third spacing pattern includes at least five electrodes arranged about 5 mm from one another.

15. The catheter kit according to claim 14, further comprising at least one additional electrode spaced apart from the third plurality of electrodes by a third gap opposite the fourth plurality of electrodes.

16. The catheter kit according to claim 15, wherein the third gap is between about 10 and 40 cm.

17. The catheter kit according to claim 12, wherein the first spacing pattern includes at least four electrodes comprising two pairs of electrodes having a spacing of about 2 mm between each of the electrodes in a pair, and each pair of electrodes are spaced apart by about 5 mm from an adjacent pair.

18. The catheter kit according to claim 9, wherein the second catheter is configured for insertion via the right femoral vein.

19. A catheter kit comprising:
a first catheter including a first electrode configuration to provide access to a plurality of first intraluminal electrophysiological monitoring and/or pacing sites via a first pathway during a procedure; and
a second catheter including a second electrode configuration to provide access to a plurality of second intraluminal electrophysiological monitoring and/or pacing sites during the procedure.

20. The catheter kit according to claim 19 wherein the second catheter is configured to provide access to the plurality of second intraluminal electrophysiological monitoring and/or pacing sites via the first pathway.

21. A catheter kit according to claim 19 wherein the second catheter is configured to provide access to the plurality of second intraluminal electrophysiological monitoring and/or pacing sites via a second pathway, that is different than the first pathway.

22. A catheter kit comprising:
a first catheter comprising a first plurality of electrodes on a distal portion of the first catheter having a spacing of 2-5-2 therebetween and comprising a second plurality of electrodes on a proximal portion of the first catheter having a spacing of about 8 mm therebetween, the first and second pluralities of electrodes having a gap therebetween of about 33 mm; and
a second catheter comprising a third plurality of electrodes on a distal portion of the second catheter having a spacing of 2-5-2 therebetween and comprising a fourth plurality of electrodes on a proximal portion of the second catheter having a spacing of about 5 mm therebetween, the pluralities of first and second electrodes having a gap therebetween of about 25 mm.

23. A catheter comprising:
a catheter body configured for insertion into the heart during a procedure, the catheter body having a first plurality of electrodes at a proximal portion thereof and a second plurality of electrodes at a distal portion thereof, wherein the first plurality of electrodes is configured to monitor electrophysiological signals and/or pace at an inter-atrial septum/HIS bundle region of a heart and the second plurality of electrodes is configured to monitor electrophysiological signals and/or pace at a right ventricle of the heart.

24. The catheter according to claim 23, wherein the first and second plurality of electrodes are spaced apart by a gap.

25. The catheter according to claim 24, wherein the gap is between about 15 and 35 mm.

26. The catheter according to claim 23, wherein the first plurality of electrodes are spaced apart from one another by a first spacing pattern and the second plurality of electrodes are spaced apart from one another by a second spacing pattern, and the first spacing pattern is different from the second spacing pattern.

27. The catheter according to claim 26, wherein the first spacing pattern includes at least six electrodes arranged about 5 mm from one another, and the second spacing pattern includes at least four electrodes comprising two pairs of electrodes having a spacing of about 2 mm between each of the electrodes in a pair, and each pair of electrodes are spaced apart by about 5 mm from an adjacent pair.

28. The catheter according to claim 26, wherein the first spacing pattern includes at least five electrodes arranged about 5 mm from one another, and the second spacing pattern includes at least four electrodes comprising two pairs of electrodes having a spacing of about 2 mm between each of the electrodes in a pair, and each pair of electrodes are spaced apart by about 5 mm from an adjacent pair.

29. The catheter according to claim 28, further comprising at least one additional electrode spaced apart from the first plurality of electrodes by a second gap opposite the second plurality of electrodes.

30. The catheter according to claim 28, wherein the second gap is between about 10 and 40 cm.
